(19) United States
(12) Patent Application Publication
(10) Pub. No.: US 2022/0081730 A1
(43) Pub. Date: Mar. 17, 2022

(54) METHOD, SYSTEM AND APPARATUS FOR RESPIRATORY TESTING

(71) Applicant: Tangen Biosciences, Inc., Branford, CT (US)

(72) Inventors: John F. Davidson, Guilford, CT (US); Zheng Xue, Madison, CT (US); Tony Joaquim, Milford, CT (US)

(73) Assignee: Tangen Biosciences, Inc., Branford, CT (US)

(21) Appl. No.: 17/533,829

(22) Filed: Nov. 23, 2021

Related U.S. Application Data
(63) Continuation-in-part of application No. PCT/US2021/045630, filed on Aug. 12, 2021.
Application No. PCT/US2021/045630 is a continuation-in-part of application No. 17/400,136, filed on Aug. 12, 2021.
Application No. 17/400,136 is a continuation-in-part of application No. 15/522,039, filed on Apr. 26, 2017.
(60) Provisional application No. 63/117,446, filed on Nov. 23, 2020.
(60) Provisional application No. 63/117,434, filed on Nov. 23, 2020.
(60) Provisional application No. 63/117,442, filed on Nov. 23, 2020.
(60) Provisional application No. 62/074,325, filed on Nov. 3, 2014.

(51) Int. Cl. C12Q 1/70; B01L 3/00

(52) U.S. Cl. CPC C12Q 1/701; A61B 10/0051; B01L 3/50215

(57) ABSTRACT

The disclosure relates to method, system and apparatus for respiratory testing. In one implementation, a method for detecting one or more pathogens causing a respiratory infection includes the steps of: (1) combining a quantity of mucous sample from a patient with a fluidic reagent to form a test reagent, the mucous sample further comprising at least one pathogen; (2) filtering the test reagent through a large volume concentrator to obtain a filtered solution; and (3) introducing the filtered solution onto a disc having a plurality of test site, wherein each of the plurality of test sites further comprises at least one agent to bind with the at least one pathogen to detect presence or absence of an infection caused by the at least one pathogen. The mucus sample may be obtained from nasal, nasopharyngeal or throat regions of the patient.

### RELATED APPLICATIONS

The disclosure claims priority to the U.S. Provisional Application No. 63/117,434, filed Nov. 23, 2020 (entitled “Method, System and Apparatus for Blood Processing Unit”), to the U.S. Provisional Application No. 63/117,442, filed Nov. 23, 2020 (entitled “Method, System and Apparatus for Respiratory Testing”); to U.S. Provisional Application No. 63/117,446 filed Nov. 23, 2020 (entitled “Method, System and Apparatus for Detection”); this disclosure to International Application No. PCT/US21/45630, filed Aug. 12, 2021 (entitled “Method, System and Apparatus for Detection”), to U.S. application Ser. No. 17/400,136, filed Aug. 12, 2021 (entitled “Method, System and Apparatus for Detection”), and to U.S. application Ser. No. 15/522,039, filed Apr. 26, 2017 (entitled “Apparatus and Method for Cell, Spore, or Virus Capture and Disruption”) which itself claims priority to U.S. Provisional Application No. 62/074,325, filed Nov. 3, 2014. The disclosures of all of the preceding applications are incorporated herein in their entirety.

### FIELD

This disclosure relates generally to method, system and apparatus pertaining to Respiratory testing of a patient for identifiable diseases. The disclosed embodiments may be used, among others, to extract samples from the upper raspatory system of a patient for diagnosis purposes.

### BACKGROUND

Nucleic acid analysis methods based on the complementarity of nucleic acid nucleotide sequences can analyze genetic traits directly. Thus, these methods are a very powerful means for identification of genetic diseases, cancer, microorganisms etc. Nucleic acid amplification technologies (NAAT) allow detection and quantification of a nucleic acid in a sample with high sensitivity and specificity. NAAT techniques may be used to determine the presence of a particular template nucleic acid in a sample, as indicated by the presence of an amplification product (i.e., amplicon) following the implementation of a particular NAAT. Conversely, the absence of any amplification product indicates the absence of template nucleic acid in the sample. Such techniques are of great importance in diagnostic applications, for example, for determining whether a pathogen is present in a sample. Thus, NAAT techniques are useful for detection and quantification of specific nucleic acids for diagnosis of infectious and genetic diseases.

Identification of pathogens via direct detection of specific and unique DNA or RNA sequences has been exploited for clinical diagnostic purposes for some time. Molecular detection technologies typically have high analytical sensitivity and specificity compared to antigen and antibody-based methods. Detection of specific genomic DNA or RNA is achieved via amplification of small unique regions of the genome via NAATs such as polymerase chain reaction (PCR, RT-PCR) as well as isothermal methods including loop mediated isothermal amplification (LAMP, RT-LAMP), nucleic acid sequence-based amplification (NASBA), nicking enzyme amplification reaction (NEAR) and rolling circle amplification (RCA), for example. In the case of PCR based amplification, the need for rapid temperature thermocycling and purified sample restricts the use of the technology to a laboratory environment and limits the minimum cost, size and portability.

LAMP, unlike PCR, does not require rapid temperature cycling and so the power demands of the instrument are much lower. This enables a low-cost alternative to the traditional lab-based PCR thermocycler. In addition, LAMP has a short time to positivity—as fast as 5 minutes for strongly positive samples and the degree of sample purity required is much lower while still having analytical sensitivity comparable or superior to PCR. In order to detect RNA, a LAMP based system requires an enzyme or enzymes that can reverse transcribe the RNA template before LAMP amplification and detection. The RT-LAMP assay can therefore be either 2-step, with the first step being a dedicated reverse transcriptase enzyme copying the RNA template into cDNA followed by the geometric LAMP amplification of the target, or preferably a single enzyme RT-LAMP process such as the LavaLAMP™ enzyme from Lucigen Inc., Middleton, Wis.

Upper respiratory tract infections are usually detected by taking swabs from the nasal, nasopharyngeal or throat and eluting the virus from them. The preparation of the RNA for detection by PCR requires further purification to remove contaminants that are less inhibitory to LAMP reactions. This enables a rapid and easy sample preparation for LAMP based assays—a requirement for simple point of care use. In the case of swab, directly eluting the virus into a suitable assay buffer and directly putting that sample into the molecular test system with a simple transfer step is enabling for point of care operation.

For a point of care device, speed and simplicity of use are requirements. No precise measuring during operation or requirements for environmental temperature and humidity are

preferred, and the reagents should ideally not require freezing or refrigerated storage. Tight temperature control, automatic fluidic staging and real time monitoring of the LAMP reaction with software to analyze the reaction and report the results to the user is preferred. Bringing the speed and sensitivity of LAMP together with an automated system that is designed to allow for operation outside of a laboratory with simple to use operating steps and room temperature reagents, is a powerful point of care combination. A single enzyme RT-LAMP system reduces assay time as reverse transcription and LAMP amplification occur simultaneously and allows for detection of RNA based pathogens including the majority of respiratory viruses such as influenza A and B, coronaviruses including SARS-CoV-2, and Respiratory Syncytial Virus (RSV).

There is a need for a system that is able efficiently to look for a panel of multiple potential virus pathogens from a single sample to provide definitive diagnosis of the common early upper respiratory symptoms including sore throat, cough, mild fever and running nose to distinguish serious infections such as Sars-CoV-2 or influenza from mild disease caused by rhinovirus or adenovirus.

### BRIEF SUMMARY

The disclosed embodiments provide, among others, rapid, accurate LAMP amplification detection with a low-cost disposable assay disk that affords a panel of 32 (or more) different pathogen targets from a single patient sample and portability, connectivity and ease of use to allow for point of care results. The SARS-Cov-2 pandemic has underscored the pressing need for rapid accurate testing outside of the laboratory setting at the point of care, with the information getting immediately the patient so they can manage their exposure to others, as well delivering the result to public health databases, so that the pandemic can be tracked, traced and controlled.

In an exemplary embodiment, the disclosure provides a system, method and apparatus for extracting samples from a patient's upper raspatory passages. Once extracted, the sample is transferred into a liquid transport medium which is then tested using a combination of sonication and panel testing to simultaneously identify presence of one or more respiratory infection in the sample.

### BRIEF DESCRIPTION OF THE DRAWINGS

Certain of the above-disclosed embodiments are illustrated in the following schematic representation. It should be noted that these representations are exemplary and non-limiting. Any modification of these exemplary embodiments may be made without departing from the disclosed principles. The illustrated exemplary figures, in which similar elements may be identified similarly, include:

FIG. 1 illustrates an exemplary respiratory assay workflow according to one embodiment of the disclosure;

FIG. 2 shows an exemplary process flow for collecting and testing a respiratory assay from a patient according to one embodiment of the disclosure;

FIG. 3A illustrates an exemplary LVC according to one embodiment of the disclosure;

FIG. 3B is an exploded illustration of the LVC of FIG. 3A;

FIG. 4 illustrates an exemplary assay disc according to one embodiment of the disclosure;

FIG. 5 illustrates an exemplary public health surveillance system according to one embodiment of the disclosure;

FIG. 6A illustrates an exemplary cross-sectional view of a detection instrument to receive a sample disc and an LVC according to one embodiment of the disclosure; and

FIG. 6B illustrates another cross-sectional view of detection apparatus illustrated in FIG. 6A.

### DETAILED DESCRIPTION

Various aspects of the invention will now be described with reference to the following section which will be understood to be provided by way of illustration only and not to constitute a limitation on the scope of the invention.

In some embodiments, an apparatus and methods for rapid isolation, concentration, and purification of microbes/pathogens of interest from a raw biological sample such as respiratory mucous is described. Samples may be processed directly from biological or clinical sample collection vessels, such as vacutainers, by coupling with the sample processing apparatus in such a manner that minimizes or eliminates user exposure and potential contamination issues. In various embodiments, the apparatus comprises a staged syringe or piston arrangement configured to withdraw a desired quantity of biological sample from a sample collection vessel. The sample is then mixed with selected processing reagents preparing the sample for isolation of microbes or pathogens contained therein. Sample processing may include liquefying or homogenizing non-pathogenic components of the biological specimen and performing various fluidic transfer operations induced by operation of the syringe or piston. The resulting sample constituents may be redirected to flow across a capture filter or membrane of appropriate size or composition to capture specific microbes/pathogens or other biological sample constituents. Additional operations may be performed including washing and drying of the filter or membrane by action of the syringe or piston. In various embodiments, sample backflow and cross-contamination within the device is avoided using one-way valves that direct sample fluids along desired paths while preventing leakage, backflow, and/or undesired sample movement.

The device may include a capture filter for retaining microbes/pathogens of interest allowing them to be readily separated from sample eluent or remaining fraction of the processed sample/waste. The capture filter may be housed in a sealable container and can further be configured to be received directly by other sample processing/analytical instruments for performing downstream operations such as lysis, elution, detection and identification of the captured microbes/pathogens retained on the filter/membrane.

The collector may comprise various features to facilitate automated or semi-automated sample processing and include additional reagents contained in at least one reservoir integrated into the collector to preserve or further process the isolated microbes/pathogens captured or contained by the filter/membrane. In various embodiments, the collector may contain constituents capable of chemically disinfecting the isolated microbes/pathogens or render the sample non-infectious while preserving the integrity of biological constituents associated with the microbe/pathogen such as nucleic acids and/or proteins that may be desirably isolated for subsequent downstream processing and analysis. The collector and associated instrument components may desirably maintain the sample in an isolated environment avoiding sample contamination and/or user exposure to the sample contents.

In various embodiments, this present disclosure describes an apparatus that permits rapid and semi-automated isolation and extraction of microorganisms such as bacteria, virus, spores, and fungi or constituent biomolecules associated with the microorganisms, such as nucleic acids and/or proteins from a biological sample without extensive hands-on processing or lab equipment. The apparatus has the further benefit of concentrating the microbes, pathogens, or associated biomolecules/biomaterial of interest. For example, bacteria, virus, spores, or fungi present in the sample (or nucleic acids and/or proteins associated therewith) may be conveniently isolated from the original sample material and concentrated on the filter or membrane. Concentration in this manner increases the efficiency of the downstream assays and analysis improving detection sensitivity by providing lower limits of detection relative to the input sample.

The sample preparation apparatus disclosed herein may further be adapted for use with analytical devices and instruments capable of processing and identifying the microorganisms and/or associated biomolecules present within the biological sample. In various embodiments, the sample collector and various other components of the system can be fabricated from inexpensive and disposable materials such as molded plastic that are compatible with downstream sample processing methods and economical to produce. Such components may be desirably sealed and delivered in a sterile package for single use thereby avoiding potential contamination of the sample contents or exposure of the user while handling. In various embodiments, the reagents of the sample collector provide for disinfection of the sample constituents such that may be disposed of without risk or remaining infectious or hazardous. The sample collector provides simplified workflows and does not require specialized training or procedures for handling and disposal.

In various embodiments, the automated and semi-automated processing capabilities of the system simplify sample preparation and processing protocols. A practical benefit may be realized in an overall reduction in the number of required user operations, interactions, or potential sample exposures as compared to conventional sample processing systems. This results in lower user training requirements and fewer user-induced failure points. In still other embodiments, the system advantageously provides effective isolation and/or decontamination of a sample improving overall user safety while at the same time preserving sample integrity, for example by reducing undesirable sample degradation.

### Exemplary Embodiments

Certain of the above-disclosed embodiments are illustrated in the following schematic representations. It should be noted that these representations are exemplary and non-limiting. Any modification of these exemplary embodiments may be made without departing from the disclosed principles.

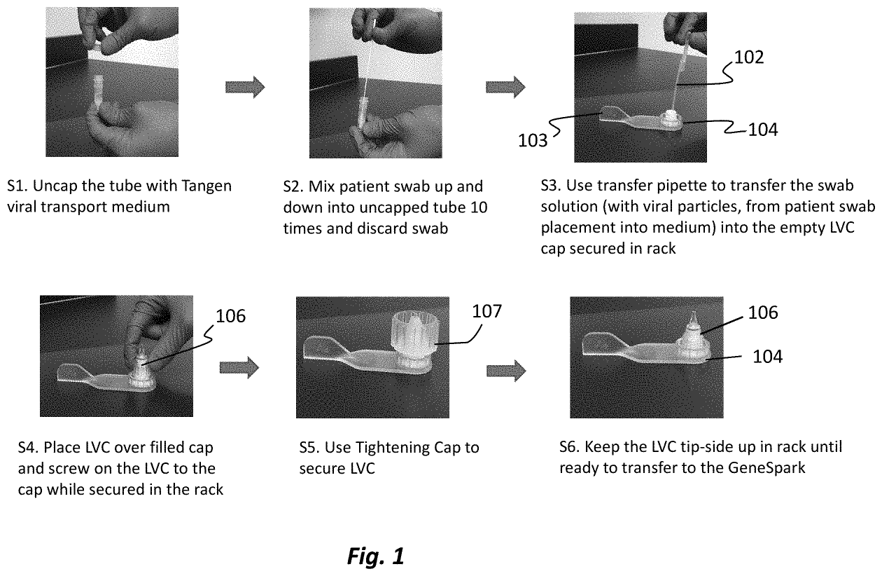

FIG. 1 illustrates an exemplary respiratory assay workflow according to one embodiment of the disclosure. At step 1 of the workflow, a tube containing viral transport medium is uncapped. The viral transport medium may comprise conventional reagents and buffers and the tube maybe pre-filled with such reagents and/or buffers. In one embodiment of the disclosure, the tube contains a pre-defined viral transport medium.

A patient's nasal mucus may be captured on a swab tip. At step 2, a swab containing the patient's nasal mucus is inserted in the uncapped tube and agitated so as to admix the extracted nasal mucus with the viral transport medium. The admixture comprises the patient's swab solution.

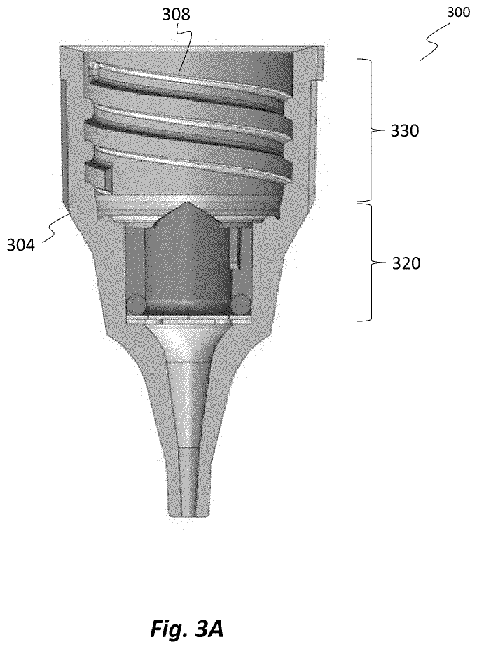

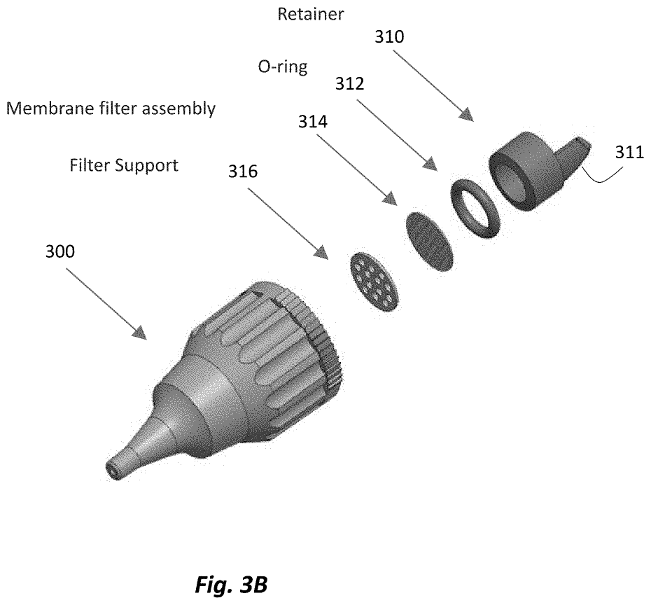

At step 3, transfer pipette 102 is used to transfer the patient's swab solution into an empty Large Volume Concentrator (LVC) cap 104. The LVC cap 104 is securely placed on rack 103 designed to receive and maintain LVC cap 104 in place. A description of the LVC is provide in relation to FIGS. 3A and 3B.

At step 4 of FIG. 1, the LVC 106 is secured to the filled cap 104 while the cap is still secured to rack 103. At step 5 of FIG. 1, tightening cap 107 is optionally used to further secure LVC 106 to LVC cap 104 through fastening. At step 6, LVC 106 is held at a tip-up position on the rack until it is ready to be transferred onto the exemplary testing device (described below).

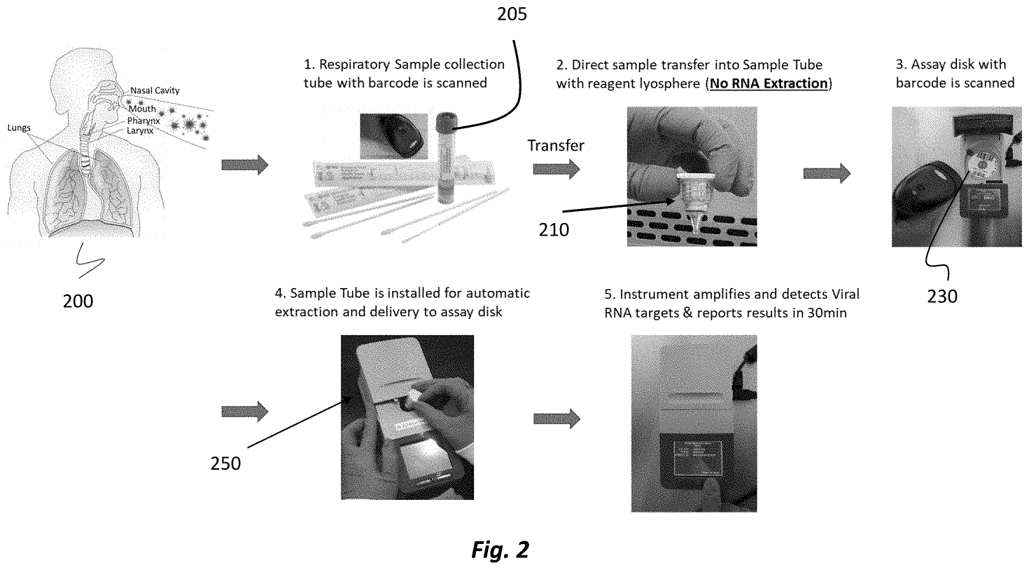

FIG. 2 shows an exemplary process flow for collecting and testing a respiratory assay from a patient according to one embodiment of the disclosure. Specifically, the upper raspatory section of patient 200 is shown expelling mucus from the patient's nasal cavity. By way of example, the mucus is shown as particles of different sizes. At step 1 of FIG. 2, respiratory sample is collected into sample tube 205, tube 205 is barcoded and the barcode on tube 205 is scanned. Sample collection may be similar to those described in FIG. 1 and use a sample collection kit according to the disclosed embodiments. An exemplary kit may include nasal passage swabs, one or more test tube and a plurality of barcoded tags for tracking the sample (as shown in FIG. 2). In one embodiment, the collection tube may comprise one or more reagents (not shown), including Tris buffer, KCl, MgSO4, BSA protein or other blocking agents, lyophilized reagents including nucleotides, DNA polymerase enzyme and Rnase inhibitor. An exemplary reagent is Lyosphere. Additional optional steps (e.g., mechanical or thermal agitation) may be implemented to prepare the sample inside the tube.

At step 2 of FIG. 2, the sample is transferred into LVC 210. In an exemplary implementation, direct sample transfer may be done with reagent Lyosphere involving no RNA extraction.

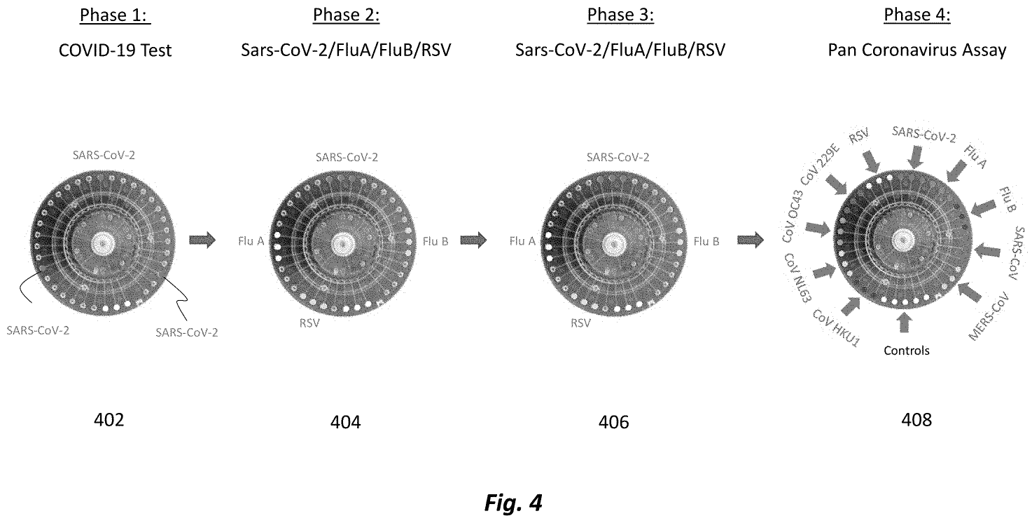

At step 3 of FIG. 2, an exemplary assay disc 230 is scanned with a barcode reader to associate the assay disk with the sample tube 205. An exemplary assay disc is described in relation to FIG. 4. At step 4 of FIG. 2, the LVC (which now contains, among others, the sample) is installed on an exemplary detection instrument 250 for automatic extraction and delivery to the assay disk.

At step 5 of FIG. 2, detection instrument 250 is activated. Detection instrument 250 may comprise sonication means and centrifugal means to timely rotate disc 230 in different directions to initiate one or more reactions within disc 230. Once activated, instrument 250 amplifies and detects viral RNA targets and reports the results.

FIG. 3A illustrates an exemplary LVC according to one embodiment of the disclosure. Specifically, FIG. 3A schematically illustrate the inside of the LVC 106 (FIG. 1), LVC 210 (FIG. 2). FIG. 3B is an exploded illustration of the LVC of FIG. 3A. With reference to FIGS. 3A and 3B, LVC 300 includes LVC tube housing 304 and threaded portion 308. An exemplary LVC tube housing 304 may receive retainer 310, O-ring 312, membrane filter assembly 314 and filter support 316. Threaded portion 308 may be used to receive an adapter. In some embodiments, threaded portion 308 may be used to couple LVC 300 directly to other components of the system.

Filter support 316 may comprise any suitable material, including inert material, to support membrane filter assembly 314. Membrane filter assembly 314 may be formed of any suitable material with holes, opening, aperture or perforation formed therein. The membrane filter size may be selected to retain pathogenic particles and component while allowing other fluid and material to pass through. O-ring 312 may be placed over membrane filter assembly 314 and filter support 316. Finally, retainer 310 may be inserted over O-ring 312 to keep the entire assembly within LVC 300. Retainer 310 may optionally comprise a notch portion 311. Notch 311 may define a sharp protrusion which extends out of a lateral plane of retainer 310 and extends towards inlet 330 (which may be threaded 308) of the LVC 300. In some embodiment, notch portion 311 may be configured to puncture a surface received at inlet 330 and threaded (or positioned) adjacent to lower portion 320 of LVC 300. As show in FIG. 3A, this entire assembly shown in FIG. 3A may be received and housed at the lower portion 320 of LVC 300.

FIG. 4 shows exemplary phases of the respiratory panels according to one embodiment of the disclosure. The discs illustrated in FIG. 4 are adapted for placement in the testing instrument 250 where the disc is received and rotated to centrifugally distribute sample received from an LVC to different location at the periphery of the disc. The different locations at the periphery of the disc may include detection assays for identifying presence (or absence) of indicator in the sample. As a result, each disc may have multiple sample detection capability. Alternatively, a disc may be configured to detect only one indicator.

Referring to FIG. 4, disc 402 is configured to detect for one or more indicators. Detection may be done by using an assay at each of wells formed on a disc. Each assay may be configured to identify one indicator. Because the disc has multiple wells, each disc can be used to detect the presence of one or more indicators. Disc 402 has three designated assays to detect presence (or absence) of SARS-CoV-2 (COVID-19). This is illustrated be rendering three locations of disc 402 as marked detection sites 402-A.

Disc 404 is configured to detect the presence of RSV, Flu B, Flu A and SARS-CoV-2 as indicated by the wells associated with each respective detection site on the disc periphery.

Disc 406 is configured to detect the presence of Sars-CoV-2, FluA, FluB and RSV as indicated by the wells associated with each respective detection site on the disc periphery.

Disc 408 is configured to detect pan coronavirus assay. It includes one or more wells devised to detect the presence of MERS-CoV, SARS-CoV, Flu B, Flu A, SARS-CoV-2, RSV, CoV229E, CoV0C43, CoVNL63 and CoV HKU1. Disc 408 also has several wells reserved for control assays.

In an exemplary application of the disclosed principles, a patient's respiratory samples are obtained and loaded onto an LVC (e.g., according to the exemplary representation of FIG. 1). A disc containing assays of interest may then be presented. The disc may include one or more assays of interest (e.g., as illustrated in relation to FIG. 4). The disc is then inserted onto the instrument as illustrated at step 230 of FIG. 2. The disc may optionally be scanned to associates the disc' assay information (e.g., though barcode) with the patient's information. The LVC which contains the patient's sample is then loaded onto the detection instrument (e.g., instrument 250, FIG. 2). Once detection instrument 250 is activated, it detects the presence of viral RNA targets and report the results (See step 5, FIG. 2).

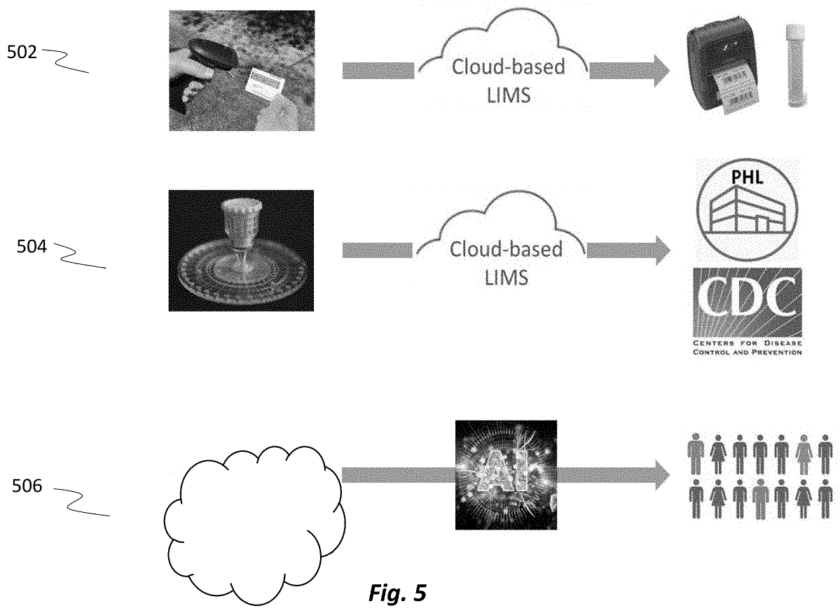

FIG. 5 illustrates an exemplary public health surveillance system according to one embodiment of the disclosure. At step 502, FIG. 5 shows accessioning of the patient demographics and triage detailed captured and transferred to the cloud. At step 504, FIG. 3 illustrates testing whereby the high priority specimen testing are tested with a field device (LVC and the assay disc shown) and kit. In one implementation, the results may be linked to laboratory information management system and transferred to public health leadership (PHL) and the Center for Disease Control (CDC). Step 506 illustrates monitoring which may combine the available information with Artificial Intelligence (AI) for long-term public health prediction and surveillance of respiratory illnesses.

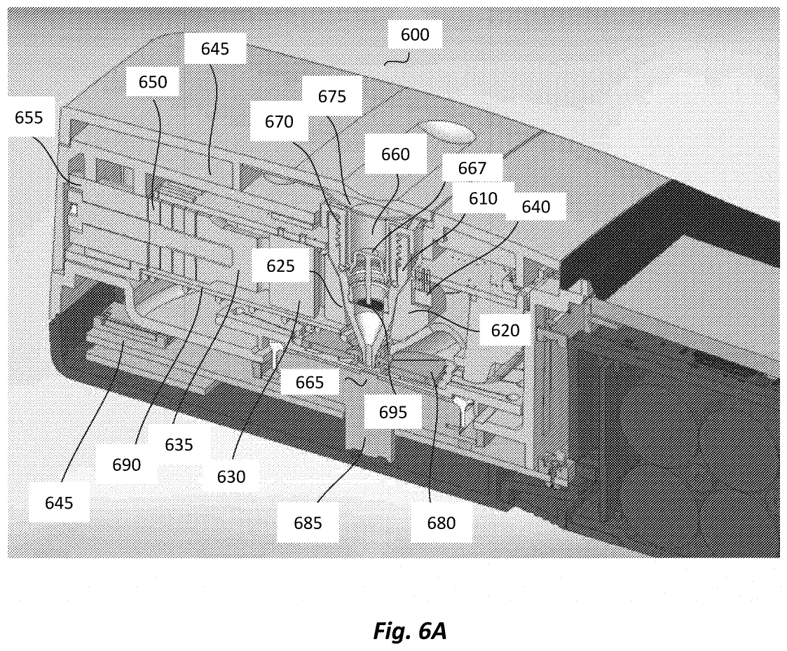

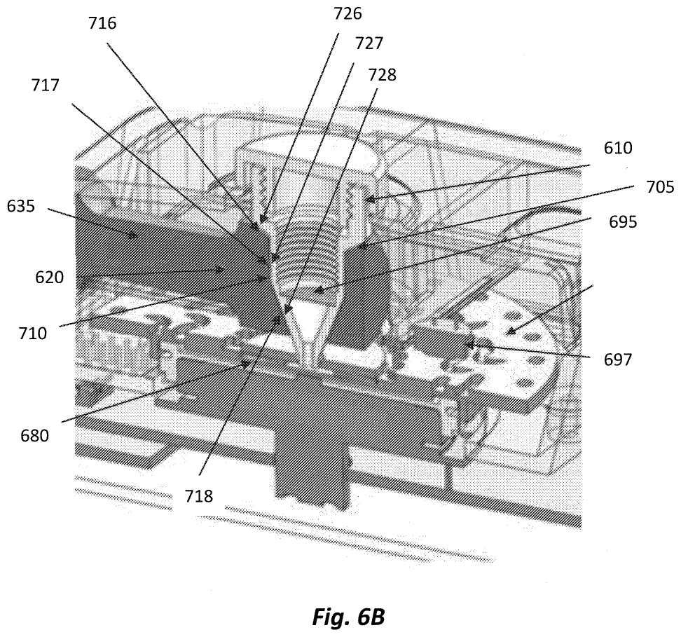

An exemplary cross-sectional view of a detection instrument according to one embodiment of the disclosure is depicted at FIG. 6A. Detection apparatus 600 of FIG. 6 is shown with sample collector 610 adapted to receive and contain a specimen or sample. Sample collector 610 may be an LVC as described and illustrated above. In various embodiments, the specimen may comprise sample obtained from a patient, such as those described herein (e.g., biomaterial, bodily fluid, urine, blood, stool, sputum, cells, tissue, spores, or other components). Various materials, reagents, buffers, analytes, or isolates may be desirably recovered from the specimen or sample including by way of example, bacteria or other microorganisms, proteins, nucleic acids, carbohydrates, chemicals, biochemicals, particles or other components present within the sample or specimen.

The sample or biomaterial may include infectious, toxic, or otherwise hazardous material that is desirably isolated within sample collector 610 in such a manner so as to minimize or eliminate exposing the user or handler of the sample collector 610 to sample constituents prior to rendering the sample constituents inactive, inert, or in a form that reduces that risk of harm or contamination. Sample collector 610 avoids unintended release of sample constituents by leakage from the sample collector 610 including preventing the escape of aerosols or particulates that might otherwise present a contamination risk to the user.

The sample or specimen may further comprise solid, semi-solid, viscous, or liquid materials. In certain embodiments, liquid or fluidic reagents (for example, buffers, water, lysis reagents, or other chemical solutions) may be added to the sample or specimen to aid in propagation of energy to disrupt or lyse the sample. Similarly, solid materials such as beads or particulates may be added to the sample or specimen to aid in disruption or lysis. Materials added to the sample may further include various reagents that facilitate sample dispersion, homogenization, emulsification, or lysis. These materials may further act to render the sample inert, inactive, or sterile. In certain embodiments added materials may chemically or physically react with released sample or specimen components to preserve or prepare the released components for downstream processing.

Detection apparatus 600 further comprises a cavitation-inducing actuator or transducer 620 configured to receive and orient the sample collector 610 in a desired position within the apparatus 600. Transducer 620 comprises a transducer interface 625 whose geometry and size are generally configured with at least a portion complementary to the sample collector 610. In various embodiments, an exterior surface contour or shape of the sample collector 610 may be configured to generally align with and/or be positioned against the transducer interface 625 such that sample collector 610 is seated or located within a portion of the transducer 620. As will be described in greater detail hereinbelow, the configuration and positioning of the sample collector 610 within the transducer 620 provides a close coupling between the sample collector 610 and the transducer interface 625 thereby permitting efficient energy transfer.

Transducer 620 may further be associated with a heater, chiller or temperature moderating element 630. In various embodiments, a heater is configured to adjustably transmit heat to the sample collector 610 either directly or indirectly. For example, a heating element 630 may comprise a controllable resistive heater embedded within or abutting against an armature 635 of the transducer 620 and capable of transmitting heat energy into the transducer 620. As the transducer armature 635 is heated this energy may further be transmitted through the interface 625 into the sample collector 610. In addition to heating means, the transducer armature 635 may be similarly configured to cool the sample as desired.

Detection apparatus 600 may further comprise a temperature sensor 640 configured to monitor the temperature of the transducer 620 and/or the sample collector 610. One or more controller boards 645 may receive signals from the temperature sensor 640 and direct operation of the heater/cooler 630 to achieve or maintain a desired temperature within the sample collector 610. In various embodiments, the combined effect of controlled temperature and energy transmission into the sample collector 610 enhances the ability of the apparatus to achieve desired agitation, lysis and/or disruption characteristics for processing of sample constituents contained within the sample collector.

Energy generation by the transducer 620 (for example, sonic or ultrasonic energy) may be provided by one or more coupled piezo devices 650 resulting in controllable vibrations or oscillation of the transducer 620 to provide energy transmission into sample collector 610. Operation of the piezo devices 650 may further be directed by the controller(s) 645 which may be configured to direct the frequency of operation piezo devices 650 to achieve the desired energy transmission into the sample collector 610. In various embodiments, the transducer 620 may be secured within the apparatus 610 and provided with a tail mass 655 of appropriate weight or configuration to generate a desired or characteristic frequency of oscillation to impart sonic or ultrasonic energy into the sample collector 610.

Sample collector 610 may comprise inlet portion 660 and outlet portion 665. The inlet portion 660 may be configured to receive a sample or specimen to be processed within the sample collector 610 and secured with a cap or cover 670 which retains the sample or specimen within the sample collector 610 while preventing the escape of solid, liquid, and/or gaseous materials from the sample collector 610. In various embodiments the cover 670 is secured to the collector 610 in a screw top, snap top, or other securing/locking configuration with sufficient engagement and retention to prevent the escape of material from the sample collector 610 including avoiding formation of aerosols outside of the sample collector 610 that may otherwise contaminate the apparatus 600 or release sample constituents potentially exposing a user to infectious or otherwise dangerous materials found in the sample or specimen.

Positive engagement between sample collector 610 and transducer 620 is maintained by a load member 675. The load member 675 may further comprise a spring, button, armature, or other mechanical or electro-mechanical device configured to impart a desired load or force on the cover 670 and sample collector 610 that urges or provides a positive engagement or coupling between sample collector 610 and transducer 620 when the sample collector 610 is suitably positioned or aligned with the transducer interface 625.

In various embodiments, sample collector 610 may be configured with the outlet portion 665 capable of delivering processed sample portions to other components of detection apparatus 600 including for example an assay plate 680. In one embodiment, assay plate 680 is a disc as described herein. Assay plate 680 may further be configured to receive the processed sample and distribute or partition the sample into one or more wells, confinement regions, or chambers associated with the assay plate 680. According to certain embodiments, the assay plate 680 may be engaged by a servo or motor 685 capable of moving or rotating the assay plate and facilitating sample distribution within the assay plate 680.

According to various embodiments, valve assembly 667 may provide controlled release of processed sample portions to the outlet portion 665 of the sample collector 610. Valve assembly or actuator 667 may be configured in a normally closed position to retain sample constituents in the sample collector 610 during at least a portion of the duration energy transfer by the transducer 620. The valve assembly 667 may then be opened according to desired processing protocols to release at least a portion on the processed sample or resulting isolates or constituents. In various embodiments, valve assembly 667 may automatically open based on achieving a desired or selected pressure within the sample collector 610. For example, sample disruption or cavitation may induce a pressure differential in the interior of the sample collector 610 causing the valve assembly 667 to open. Additionally, heat generated in the sample collector (e.g., LVC) interior may cause the valve assembly 667 to open at a selected temperature or temperature range. In another embodiment, gas or vapor generated in the sample collector may cause the valve assembly 667 to open upon achieving a selected pressure or pressure range within the sample collector 610. Gas or vapor generated in the sample collector interior may result from reagents added to the sample collector or mixed with the sample. For example, reagents for generating carbon dioxide, chlorine, chlorine dioxide, nitrogen, or other gases or vapors may be used to actuate the valve assembly 667 and thereby release processed sample constituents in a controlled manner.

One or more filters (or membrane filters) 695 may be integrated into the sample collector 610. As discussed in relation to FIG. 3B, the filter(s) may aid in sample separation and/or isolation to retain selected materials within the sample collector 610 while permitting the passage of other materials. For example, sample constituents such as cells, tissue, and lysed residual materials may be desirably retained in the sample collector 610 by filters 695 while allowing the selective passage of desired sample isolates such as bacteria, viruses, nucleic acids, carbohydrates, and/or proteins. Filters 695 may further have chemical compositions or chemical moieties disposed thereon for selectively retaining various sample materials and may be used to capture and/or separate desired constituents as will be appreciated by those of skill in the art.

Detection apparatus 600 may further include one or more assay plate heating/cooling elements 690 to maintain the assay plate 680 at desired temperature ranges. Configurable heating and/or cooling in this manner may aid in performing sample reactions using various reagents and protocols. For example, processed sample received from the sample collector 610 may comprise concentrated and/or purified nucleic acids to be subjected to polymerase chain reaction or probe-based nucleic acid detection techniques within the assay plate 680 for detection and/or identification of selected sample constituents.

FIG. 6B illustrates another cross-sectional view of detection apparatus 600 depicting an exemplary configuration and engagement between the sample collector 610 and the transducer 620. Transducer 620 employs an innovative structure including an at least partially or substantially tapered, infundiblular or conical recess/cavity 705 associated with the armature 635 that is capable of receiving or coupling with sample collector 610. In various embodiments, the recess or cavity 705 of the transducer 620 is dimensioned and/or shaped in a manner that permits sample collector 610 to be positioned within or in proximity to the recess 635 whereby a sidewall or portion 710 of the sample collector 610 engages with the transducer 620 at the interface 625. In various embodiments, a close coupling between the transducer 620 and sample collector 610 is achieved by forming the recess 635 of the transducer 620 to house or contain a portion of the sample collector 610 such that sample collector 610 is at least partially inserted into or resides within the recess 635. In various embodiments, transducer interface 635 may comprise a plurality of surface contours, curvatures, or angles (exemplified by elements 716, 717, 718) that align with or are complimentary to sidewall surfaces, curvatures, or angles of the sample collector 610 (exemplified by elements 726, 727, 728). In various embodiments, configuration of the transducer 620 with an at least partially or substantially tapered, infundiblular or conical recess 705 desirably improves energy transmission between the transducer 620 and the sample collector 610.

An advantage provided by the at least partially or substantially tapered, infundiblular or conical transducer design of the present teachings is that lower frequency energy or vibrations may be efficiently transmitted into the sample collector 110. Conventional ultrasonic horns are typically configured with a relatively small area for engagement between the horn and the surface into which energy is transmitted. Such configurations may be necessary in part to ensure sufficient propagation of the ultrasonic energy and consequently provide limited or highly focused energy transmission into the sample. Such modes of energy transmission may impose significant limitations on how much of a sample may receive the energy. In applications where a sample is to be mixed, disrupted, or lysed, the relatively small or limited contact surface between the ultrasonic horn and the sample collector results in potentially reducing the overall volume or amount of sample that can be processed and may further result in incomplete or ineffective sample mixing, disruption or lysis.

In accordance with an exemplary embodiment, an innovative transducer design overcomes the limitations of conventional transducer designs increasing the overall surface engagement or contact between the transducer 620 and the sample collector 610. The close coupling of the transducer 620 with the sample collector 610 along or about one or more surfaces, such as provided by complimentary and at least partially or substantially tapered, infundiblular or conical designs, desirably increases the overall amount of contact between the two components and provides for improved energy transmission into sample collector 610. Consequently, operations including for example, sample mixing, disruption, or lysis can be performed more efficiently, with less power, and/or more uniformly.

In certain embodiments, due at least in part to the improved or efficient energy transfer between the transducer 620 and the sample collector 610, sample processing operations such as cellular lysis or disruption to break down or disperse the sample constituents can be achieved without the use of beads or other particulates added to the sample for purposes of enhancing the efficiency of these processes. Avoidance of beads and other particulates also desirably reduces costs, simplifies processing protocols and avoids potential clogging of filters or membranes that may be used in sample processing.

An embodiment of the disclosure relates to a kit for identifying the presence of one or more disease-causing pathogens in a patient's upper respiratory system. An exemplary kit includes a plurality of swabs for collecting mucous samples from a patient, at least one respiratory sample collection tube, reagents for admixing with the collected ample, an assay disc. The reagents may comprise Tris buffer, KCl, MgSO4, BSA protein or other blocking agent, lyophilized reagents including nucleotides, DNA polymerase enzyme and Rnase inhibitor. The disc may comprise one or more detection sites. Each detection site may comprise a respective assay and reagents that bind with a predefined pathogen, and reagents for selective amplification of the predetermined pathogen.

While the principles of the disclosure have been illustrated in relation to the exemplary embodiments shown herein, the principles of the disclosure are not limited thereto and include any modification, variation or permutation thereof.

What is claimed is:

1. A method for detecting one or more pathogens causing one or more respiratory infections, the method comprising:
combining a quantity of mucous sample from a patient with a fluidic reagent to form a test reagent, the mucous sample further comprising at least one pathogen;
filtering the test reagent through a large volume concentrator to obtain a filtered solution; and
introducing the filtered solution onto a disc having a plurality of test site, wherein each of the plurality of test sites further comprises at least one agent to bind with the at least one pathogen to detect presence or absence of an infection caused by the at least one pathogen;
wherein the mucus sample is obtained from nasal, nasopharyngeal or throat regions of the patient.

2. The method of claim 1, wherein the fluidic reagent comprises one or more of assay buffers, water, lysis reagents.

3. The method of claim 1, wherein the fluidic reagent excludes RNA extraction.

4. The method of claim 2, wherein the reagent comprises lyophilized reagents including nucleotides, DNA polymerase enzyme, Rnase inhibitor.

5. The method of claim 1, wherein the step of filtering the test reagent further comprises exerting external energy to the test reagent.

6. The method of claim 1, wherein the eternal energy comprises one or more of mechanical, thermal or sonication energy.

7. The method of claim 1, wherein the step of filtering the test reagent further comprises removing particles exceeding a predefined size threshold.

8. The method of claim 1, wherein the reagent comprises one or more of Tris buffer, KCl, MgSO4, BSA protein or other blocking agent.

9. A system to detect presence of one or more pathogens in a sample, the system comprising:
a large volume concentrator (LVC) to receive a quantity of reagent test mixture, the LVC further comprising: a filter support, a membrane, a retainer and a threaded portion, wherein the membrane is configured to remove at least one particle from the reagent test mixture;
a transducer to exert energy to the LVC; and
an assay disc having a plurality of test site, wherein each of the plurality of test sites further comprises at least one agent to bind with the at least one pathogen to detect presence or absence of an infection caused by the at least one pathogen.

10. The system of claim 9, wherein the sample defines a mucus sample obtained from nasal, nasopharyngeal or throat regions of a patient.

11. The system of claim 9, wherein the reagent test mixture further comprises one or more of assay buffers, water, lysis reagents.

12. The system of claim 11, wherein the reagent test mixture comprises lyophilized reagents including nucleotides, DNA polymerase enzyme, Rnase inhibitor.

13. The system of claim 9, wherein the transducer provides one or more of mechanical, thermal or sonication energy to the LVC.

14. The system of claim 9, wherein the step of filtering the test reagent further comprises removing particles exceeding a predefined size threshold.

15. The system of claim 9, wherein the reagent comprises one or more of Tris buffer, KCl, MgSO4, BSA protein or other blocking agent.
